(19) United States
(12) Patent Application Publication
(10) Pub. No.: US 2014/0018828 A1
(43) Pub. Date: Jan. 16, 2014

(54) VARIABLE FRICTION BUCKLE TIGHTENING SYSTEM WITH FRICTION INDICATOR

(71) Applicants: Seth Arnold Foerster, San Clemente, CA (US); David Trottingwolf Aldridge, Laguna Hills, CA (US)

(72) Inventors: Seth Arnold Foerster, San Clemente, CA (US); David Trottingwolf Aldridge, Laguna Hills, CA (US)

(73) Assignee: DALLEN MEDICAL, INC., San Clemente, CA (US)

(21) Appl. No.: 13/476,970

(22) Filed: May 21, 2012

Related U.S. Application Data
(60) Provisional application No. 61/487,904, filed on May 19, 2011.

(51) Int. Cl. A61B 17/03

(52) U.S. Cl. 606/151

(57) ABSTRACT

A method for repairing separated tissues utilises a tissue tensioner including a locking member, a band, a frame, and a restraining member. One end of the band is attached to a first end of the frame. Tension is applied to the band so that it moves freely between spaced engaging surfaces on the locking bar and a second end of the frame, while a restraining member restrains the locking member to a non-engaging position. Application of a predetermined level of tension on the band causes the restraining member to move to a non-restraining orientation, thereby in turn causing the locking member to move so that its engaging surfaces engage the engaging surfaces of the frame, clamping the band. An alarm is triggered to notify a practitioner that the predetermined level of tension has been achieved and that the restraining member has thus moved to the non-restraining orientation.

This application claims the benefit under 35 U.S.C. 119(e) of the filing date of Provisional U.S. Application Ser. No. 61/487,904, entitled Variable Friction Buckle Tightening System With Friction Indicator, filed on May 19, 2011. This application is also related to co-pending and commonly assigned U.S. application Ser. No. 12/858,332, entitled Low Friction Buckle Tightening System and Methods, filed on Aug. 17, 2010. Both applications are expressly incorporated herein by reference, in their entirety.

### BACKGROUND OF THE INVENTION

The present invention is related to the general surgical repair of separated body tissues, and more particularly to internally fixating and stabilizing such body tissues, specifically bones.

Buckles are commonly used to adjust the effective length of a strap. Such strap/buckle combinations are commonly used on seatbelts, helmet chin retainers, and shoulder straps. The adjustability of the buckle makes the strap much more functional in these applications. Seatbelts can be adjusted to fit differently sized people, helmets can be cinched tightly and then taken off, and backpacks can be worn by differently sized people because the shoulder straps adjust.

There are applications where a buckle may be used in combination with a strap when re-adjustment is not as critical as the first adjustment. This would be an application where the first length adjustment of the strap is critical and closely tied to a specific tension in the strap. Such a feature adds cost and complexity to the buckle, but in certain applications, the additional cost and complexity may be justified by its added benefits.

There are many fastener devices that trade off removability of the fastener for functionality. Screws, for example, can attach objects to gypsum board and come out easily if needed. Unfortunately, however, screws don't hold very well in gypsum board. Toggle bolts don't come out easily, but hold objects rather well to gypsum board. So toggle bolts overcome a screw's lack of holding power in gypsum board at the expense of ready removability.

In the realm of fasteners, screws have insufficient surface area to work well in gypsum board. This deficiency ultimately resulted in the invention of the toggle bolt. Similarly, friction is sometimes excessive in the operation of a common buckle, and therefore unsuitable for certain applications. Accordingly, in those applications, a modified buckle that adds some user complexity and sacrifices the easy re-use of the buckle may be preferable.

Consider the case of tying down a cargo load. A strap with a buckle is considered in many cases as being unable to apply sufficient tension to the strap to make the load stable. Many users resort to a complicated ratcheting system that can easily over-tension the load and damage it. Most light cargo loads would benefit from a buckle system that has double the tensioning abilities of the common buckle. This would add some user complexity, but not the user complexity, or cost, realized in a strap ratcheting system.

### SUMMARY OF THE INVENTION

A dynamic tissue holding device for dynamically holding two tissue portions in contact with one another comprises a band adapted for extending about the tissue portions to be held together, wherein the band has a first end for attachment to a first end of a frame and a second end for attachment to a second end of a frame. The band establishes a path of tension along its length and extending linearly between the two ends of the band. The device further comprises a locking member, a mating surface disposed on the frame first end, and a restraining member situated to keep the locking member in a spaced relation to the mating surface. The restraining member is constructed to move to a non-restraining orientation when a predetermined level of tension has been achieved in the band. A tension indicator is also provided which is triggered when the restraining member moves to its non-restraining orientation.

In certain embodiments, the restraining member comprises a ribbon having a point of weakness, preferably a notch, designed therein which is adapted to break at a predetermined tension level. The ribbon is attached between the frame and the locking member, and acts to prevent movement of the locking member toward the mating surface when intact. Its fracture at the point of weakness when applied tension thereto reaches or exceeds the predetermined tension level permits the locking member to move from its initial position, spaced from the mating surface, to a second position engaging the mating surface. More particularly, the ribbon supports the locking member in its initial position, spaced from the mating surface, when intact, and its fracture at the point of weakness when applied tension thereto reaches or exceeds the predetermined tension level permits the locking member to move along a cam surface to a second engaging position.

In certain embodiments, the restraining member comprises a removable pin. In other embodiments, the frame further comprises a detent and the locking member further comprises a ball engageable with the detent, wherein when the ball and the detent are engaged, they comprise the restraining member. In these embodiments, the frame is adapted to distort sufficiently, upon application of tension thereto at the predetermined level, to dislodge the ball from the detent, so that the locking member is unrestrained and free to move from its initial position to a second engaging position.

The tension indicator, in certain embodiments, comprises an electrical circuit which passes a voltage through the restraining member and a sensor for detecting when the voltage changes as a result of the restraining member moving to its non-restraining orientation. The sensor activates a visual or audible alarm. In other embodiments, the tension indicator comprises a mechanical member which is connected to the restraining member. In certain of these mechanical embodiments, the mechanical member comprises a flag which undergoes a visible change when the restraining member moves to its non-restraining orientation. This visible change may comprise the flag detaching from the frame when the restraining member moves to its non-restraining orientation, resulting in a movement thereof which can be visually detected. The flag may comprise a base, a post, and a tab, the base functioning to connect the flag to the frame and the post raising the tab from the base for ready visibility to a practitioner. The base may comprise fingers for attaching the base to the frame in every degree of freedom except for one. That one degree of freedom not addressed by the attachment of the base to the frame is addressed by the restraining member, such that when the restraining member moves to its non-restraining orientation, the base becomes unstable and detaches from the frame.

In another aspect of the invention, there is disclosed a method of repairing separated tissues using a tissue tensioning device comprising a locking member, a band having first and second ends, a frame having opposed first and second ends, and a restraining member, wherein the first end of the band is attached to the first end of the frame. The method comprises a step of applying tension to the second end of the band so that the band moves freely between spaced engaging surfaces on the locking bar and the second end of the frame, respectively, while the locking member is restrained to a first non-engaging position by a restraining member. A further step comprises moving the locking member to a second position wherein the engaging surfaces on the locking member and the second side of the frame are sufficiently engaged to clamp the locking member in place, the moving step being performed by applying a predetermined level of tension on the band, so that the restraining member moves to a non-restraining orientation. A still further step comprises triggering an alarm to notify a practitioner that the predetermined level of tension has been achieved and that the restraining member has thus moved to the non-restraining orientation.

In certain methods, the triggering step comprises changing an electrical condition in a circuit and thus activating a visual or audible alarm. In other methods, the triggering step comprises changing a state of a mechanical member relative to the device, so that the practitioner can see the changed state of the mechanical member and know that the predetermined tension level has been achieved.

The invention, together with additional features and advantages thereof, may best be understood by reference to the following description taken in conjunction with the accompanying illustrative drawings.

### BRIEF DESCRIPTION OF THE DRAWINGS

FIG. 1 is a top view of a buckle for tensioning a strap;

FIG. 1a is a cross-sectional view taken along lines A-A of FIG. 1;

FIG. 2 is an isometric view of a sternum which has been cut into two halves, illustrating two different approaches for securing the sternal halves together for healing;

FIG. 3a is a side view of one embodiment of the present invention;

FIG. 3b is a side view similar to FIG. 3a, showing the FIG. 3a embodiment after a ribbon securing the lock bar 2 has been severed;

FIG. 4a is a side view of another embodiment of the present invention;

FIG. 4b is a side view similar to FIG. 4a, showing the FIG. 4a embodiment after a pin has been withdrawn;

FIG. 5a is a side view of yet another embodiment of the present invention;

FIG. 5b is a side view similar to FIG. 5a, showing the FIG. 5a embodiment after the lock bar has been dislodged from its initial position;

FIG. 6a is an isometric view of still another embodiment of the present invention;

FIG. 6b is a side view showing portions of the embodiment shown in FIG. 6a; and

FIG. 6c is a side view similar to FIG. 6b showing the FIG. 6a embodiment after the notch 64 has been fractured.

### DESCRIPTION OF THE PREFERRED EMBODIMENT

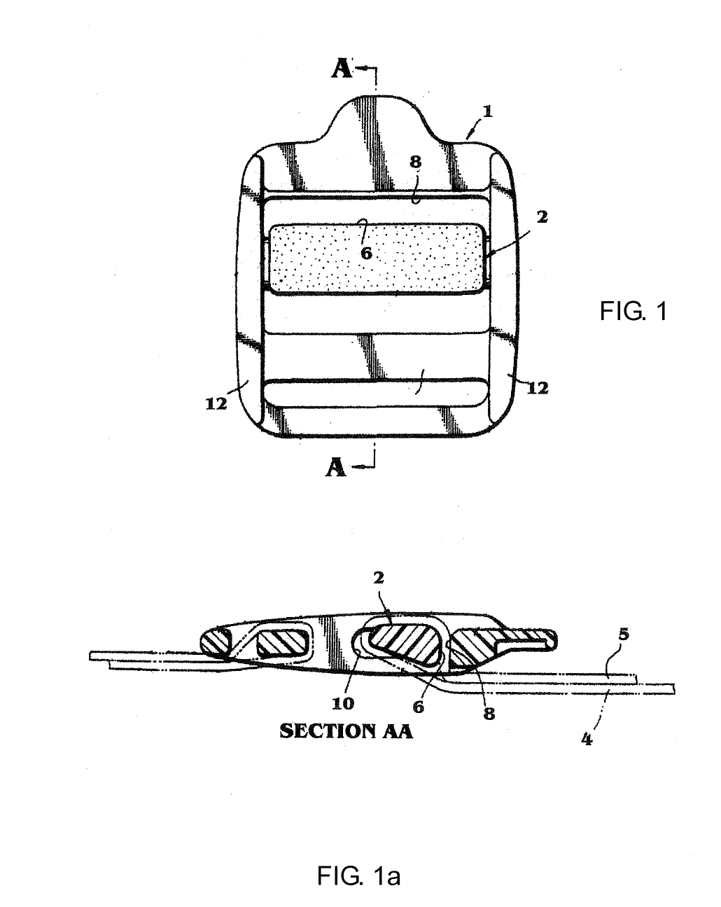

Referring now more particularly to the drawings, there is shown in FIGS. 1 and 1a a common buckle 1, which includes a lock bar 2 about which a strap having strap tails 4 and 5 extends. A surface 6 of the lock bar 2 moves away from a surface 8 on the buckle 1 when the strap tail 5 is tensioned. As the strap tail 5 is tensioned, the strap tail 4 is pulled into an interface between surfaces 6 and 8. When the strap tail 5 is released, the strap tail 4 holds the dominant tension, which pulls the lock bar 2 so that the strap is pinched between the surfaces 6 and 8. This is the locked position of the buckle 1. Lock bar 2 is held in position by tabs on both of it ends, which ride in a slot 10 which is cut out of a frame 12. The slot 10 is critical in the operation because it holds the surface 6 on the lock bar 2 so that it correctly pinches the strap up against the surface 8. The variability of the tension in strap tails 4 and 5 causes the lock bar to move in the slot 10. The lock bar naturally wants to even out these tensions by rotating in the slot 10. Flats are milled in the ends of the lock bar 2 to ride in the slot 10, so that such rotation does not happen.

The reality of lock bar 2 movement in response to tensioning of the strap tail 5 is minimal. It might be more accurate to say that the tensioning of the strap tail 5 serves to relieve some pressure on the strap in the 6/8 surface interface so that strap movement is enabled. But strap movement is accomplished only when significant friction is overcome. If there were a way to further move the lock bar 2 so that no pressure is put on the strap in the 6/8 surface interface, twice the tension would be realized in the strap 4. A common buckle on transfers 1/10 of the tension placed on the strap end 5 to strap end 4 due to the pressure put on the strap in the 6/8 surface interface. When the lock bar is held back such that there is no pressure in the 6/8 surface interface, approximately ⅕ of the tension placed on the strap end 5 is realized in strap 4.

These mechanics define the objective of this invention. The present invention comprises a mechanism that can hold the lock bar 4 so that the surface 6 is sufficiently away from the surface 8 so that no substantial friction is imparted onto the strap by those surfaces. This mechanism exists as such while the buckle is being tensioned, so that tensioning is easy. This same mechanism is able to be released so that when desired, the surface 6 is allowed to float into the surface 8, as happens in a common buckle.

The mechanism can operate in two ways. One way is to place an object between the surfaces 6 and 8 which would serve to hold them apart. This object is in place while tensioning, and then removed after the desired tension is achieved. A more advanced mechanism may sense the tension in the strap and pull out the object when the desired tension is achieved. The other mode of operation is to pull on the lock bar 2 with a tension member so that the surface 6 is held away from the surface 8. This tension member holds the surface 6 away from the surface 8 during tensioning of the strap and the is removed once the desired tension is achieved. A more advanced mechanism would sense tension in the strap and automatically release tension in the tension mechanism once the desired tension has been realized. Such an advanced mechanism may be simply a ribbon with a notch in it. The notch would be designed to fail at a specific tension that would translate to the desired tension in the strap.

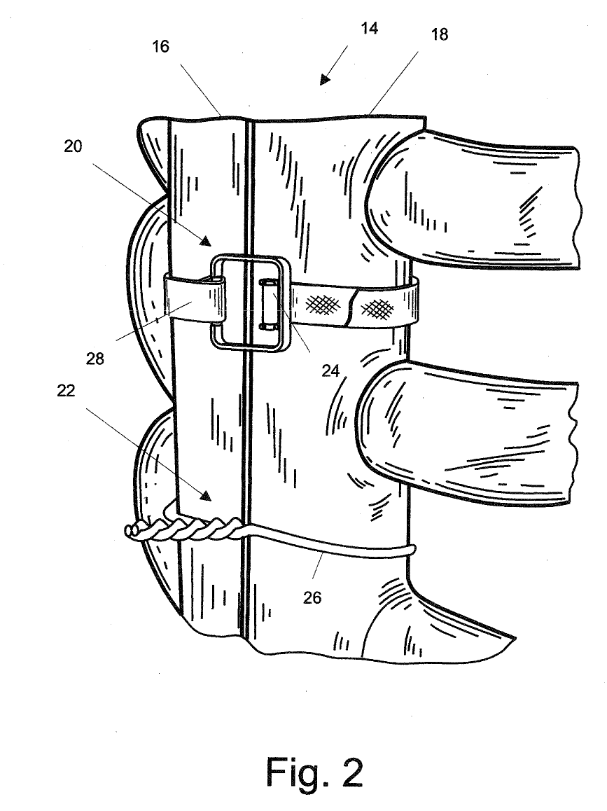

FIG. 2 shows an application for a single use buckle. Pictured is a sternum 14 that has been cut down the middle (osteotomy) for surgical access to the chest cavity. This type of osteotomy is commonly performed in open heart procedures that require surgical access to heart valves and heart arteries. The sternum is cut into two halves 16 and 18 and the chest is separated to gain access to internal organs. After surgery, closure of the sternum must be performed to ensure proper healing. Two different approaches for securing the sternal halves 16, 18 for healing are illustrated. In the prior art, as illustrated, a wire 26 may be wrapped around the sternal halves, as shown, and twisted at 22 to secure it in place.

However, wires have many complications when used in this capacity. Wires 26 are thin and cut into the bone. The forces realized by twisting the wires are highly variable. The twisted wires 22 can untwist when the patient coughs. The wires can cyclically fail, fall off the sternum, and migrate to puncture arteries and organs. All of these failures may be addressed by using a buckled band 20 instead. The buckled band 20 comprises a band 28 wrapped around the sternal halves 16, 18 and terminating into a buckle 24. The band 28 is tightened by means of cinching the band through the buckle 24. Buckle band 20 is ideal for the invention as it is a single use buckle. The band 28 can be cinched through the buckle 24 in a reduced friction environment until a programmed force triggers a mechanism within the buckle and the buckle locks down onto the band.

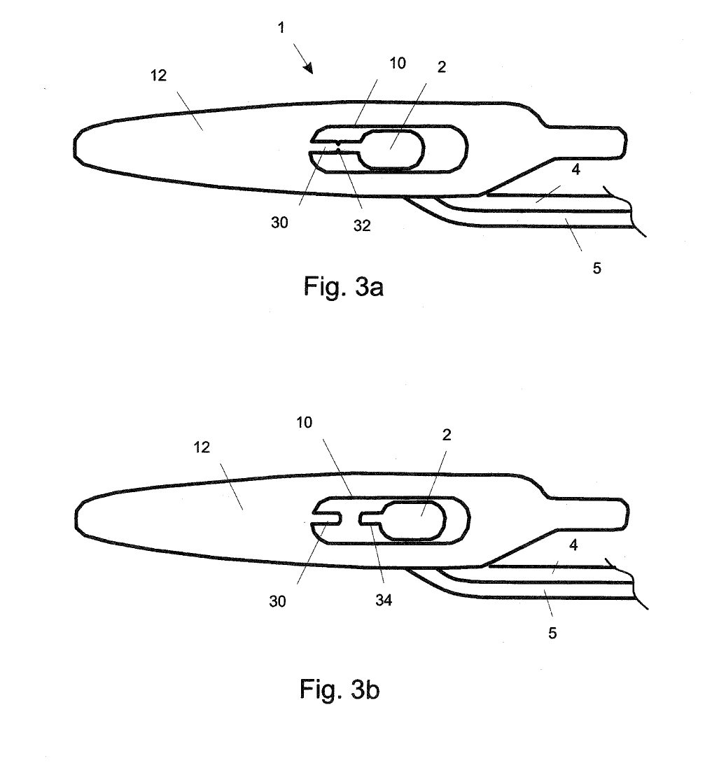

FIG. 3a shows a side view of a buckle 1 similar to that shown in FIGS. 1 and 1a, but modified as described below. The side of the buckle frame 12 is shown, as is the side view of the lock bar 2 with band tails 4 and 5 exiting the buckle 1. A new element, ribbon 30, is shown to connect the lock bar 2 to the buckle frame 12. The ribbon 30 is placed on both ends of the lock bar 2 so that it holds the clamping surfaces 6 and 8 away from the band. This reduces the friction in the buckle, making the band much easier to cinch within the buckle. When enough cinching force is applied to the band, the lock bar 2 pulls sufficiently had on the ribbon 30 to break a notch 32 contained within the ribbon as shown in FIG. 3b as ribbon pieces 30 and 34. After this time, the lock bar 2 is free of the ribbon 30, now broken, and can function as it would in the buckle shown in FIGS. 1 and 1a.

Ribbon 30 allows the clamping surfaces 6 and 8 to be far enough away from each other so as not to impart friction onto the band. The tension realized in the band tail 5 is equal to the tension in the band tail 4 minus the friction internal to the buckle. Decreasing the friction internal to the buckle, as the inventive embodiment does, increases the amount of tensioning force that can be translated from the band tail 4 to the band tail 5.

All of the foregoing embodiments illustrate buckles containing bands. There are many fasteners that rely on similar mechanisms for securing an elongated member around an object. This invention applies not only to buckles, but to any mechanism that employs a movable clamping element that is managed by the tension in the cinching tail, because the retaining concepts invented in connection with the illustrated embodiments, for the movable clamping element, are broadly applicable. Such a fastener may employ any number of different elongated members, such as bands, ropes, sutures, monofilaments, cables, braided structures, woven structures which could be made out of plastics, metals, re-absorbable plastics, ceramics, textiles, crystalline materials, and amorphous materials.

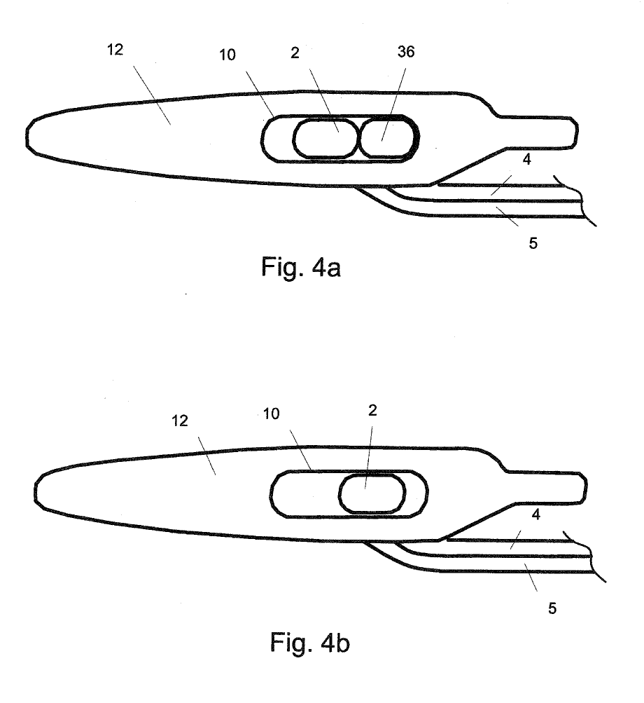

FIGS. 4a and 4b illustrate another embodiment which utilizes a pin 36 instead of a ribbon for retaining the lock bar 2. The pin 36 is placed on both ends of the lock bar 2 such that it holds clamping surfaces 6 and 8 away from the band. This reduces the friction in the buckle, thus making the band much easier to cinch within the buckle. When the desired tension is realized in the band tail 5 by pulling on the band tail 4, the pins 36 are removed, as shown in FIG. 4b, to allow the lock bar 2 to function in the slot 10 as it does in a normal buckle (FIG. 1).

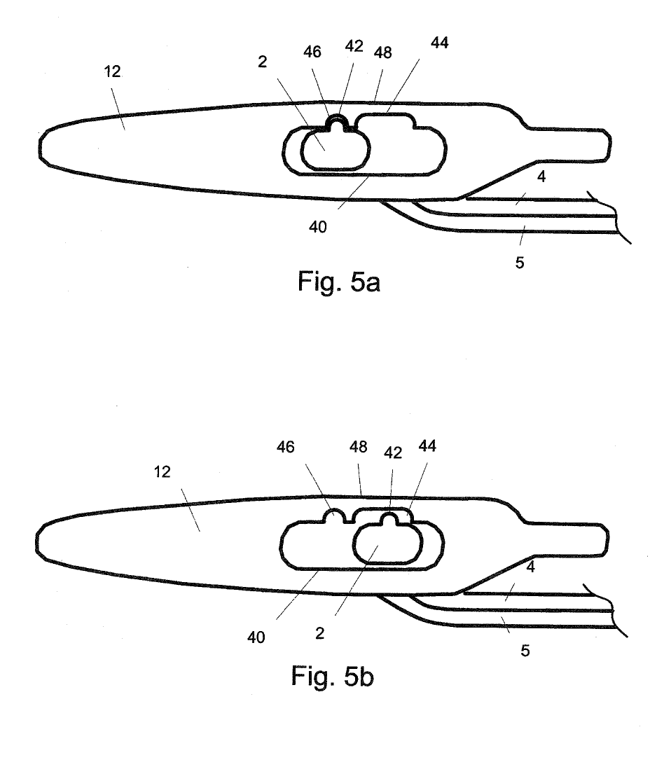

FIGS. 5a and 5b show still another embodiment of the present invention, which utilizes a concept similar to a ball detent to retain the lock bar 2 while tensioning the band tail 4. Only one side of the buckle is shown. However, the other side thereof is a mirror image of the visible side. A ball 42 is attached to the lock bar 2 for the purposes of residing in a detent 46. The lock bar 2 slides in a slot 40, which has the detent 46 and another detent 44. The top of the slot 40 includes a member 48, which is able to flex. When tensioning the buckle, increased tension loads the lock bar 2 so that it distorts the member 48. At the desired tension, the member 48 is designed to flex so that the ball 42 is able to slide out of the detent 46 and into the detent 44 as is shown in FIG. 5b. The detent 44 is long, thereby allowing the lock bar 2 to slide as it would in a normal buckle. This allows the buckle to operate normally once the lock bar has moved out of the detent 46 and into the detent 44.

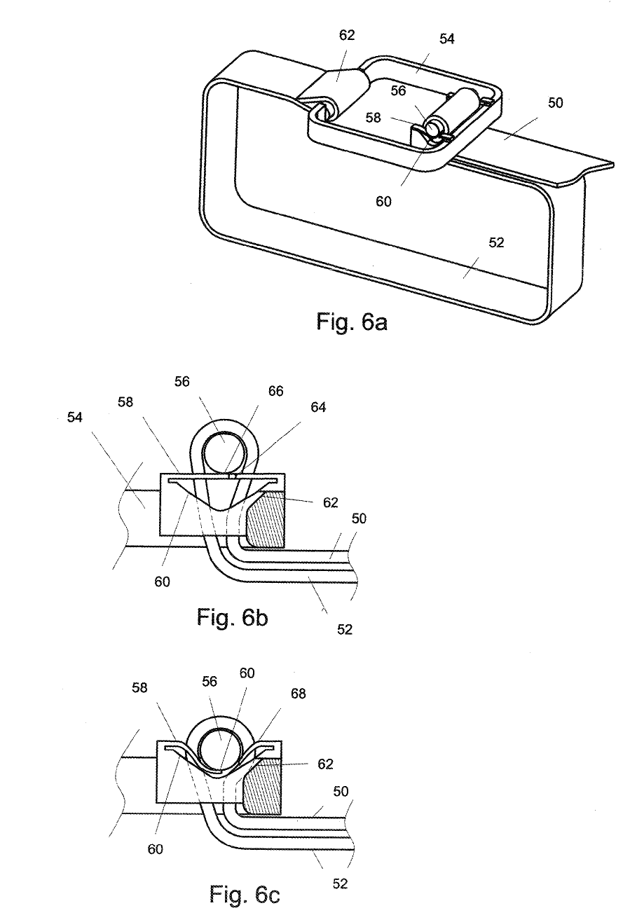

Still another embodiment is illustrated in FIGS. 6a-6c. This embodiment shows how the principle of temporarily retaining the lock bar for the purpose of tensioning can be achieved with the disclosed mechanisms outside of the typical slot/lock bar buckle design. The basic operating principle of a buckle require the presence of a floating locking member, a mating locking surface attached to a structure, a means within this structure to contain movement of the floating locking member so that it only moves into and away from the mating locking surface, and a means to keep the floating locking member from rotating. As stated in these terms, this temporarily retains the floating locking member away from its mating surface.

FIG. 6a shows a buckle that has a lock bar or pin 56 functioning as its floating locking member, surface 62 functioning as its mating locking surface on a buckle structure or frame 54, a cam surface 60 for containing movement of the pin 56, and a ribbon 58 to keep the pin 56 from rotating (see FIG. 6b for pin 56 weld location 66 to the ribbon 58). Pulling on a band end 50 of a band 52 causes the pin 56 to ride up the cam surface 60, thereby temporarily releasing its hold on the band and allowing cinching of the band.

FIG. 6b shows the pin 56 being held off of the mating locking surface 62 by means of the ribbon 58 which contains a weak point such as a notch 64. It is clear that the pin 56 is being held sufficiently away from the mating locking surface 62 so as to not have any associated engagement friction. In being structured in this way, the band tail 50 can easily tension the band 52. When a desired tension has been realized, sufficient forces are imposed on the ribbon 58 so that the notch 64 fails.

FIG. 6c shows what happens after the notch 64 fails. The ribbon 58 breaks into two pieces, leaving a ribbon remnant 68. The pin 56 rides down the cam surface 60 to settle against mating locking surface 62 through the band. Now the buckle operates as it would without the ribbon—pulling on the band tail 50 pushes the pin up the cam surface to allow the pin 56 to move away from the mating locking surface 62, allowing the band 52 to move through the buckle.

Structured as described above, this invention provides a means to allow a buckle to significantly increase its tensioning performance. Breakaway mechanisms allow this increase in performance to exist only for the first tensioning. Pin-type mechanisms, on the other hand, may be reused, allowing the repeated advantage of tensioning without the binding friction found in normal buckles. There are also ways that the ball-detent mechanism may be reset as well.

It is clear now that the present invention provides a means to reduce friction in the buckle during the buckle's initial tensioning. The FIGS. 5 and 6 embodiments contain mechanisms that are based on tension in the band. These mechanisms provide for low friction in the buckle path, but then switch to a higher frictional state when a specific tension has been realized in the tensioning band. This ability to impose a specific tension to the function of the buckle has a benefit that is, in itself, an invention. The inventive aspect of this dynamic is that the buckle clearly identifies when a specific tension has been realized in the band.

Specific tensions are not commonly monitored in fasteners. Wood screws cannot indicate that they are about to strip the wood off its threads, package banding systems cannot indicate that the packages are held together tightly enough, and sternal repair fasteners cannot indicate to the surgeon that the bones are being held together sufficiently to achieve optimum healing. As noted above, FIG. 2 shows the sternum 14 on which an osteotomy has been performed in the course of a common open heart procedure. The buckle 20 embodiment does not tell the surgeon when appropriate tensions have been applied to the band 24 such that optimum healing may commence. The surgeon relies largely on his sense of feel in holding and tensioning the band 24 to determine whether or not sufficient tensions exist in the band. The other illustrated embodiment of FIG. 2, comprising twisted wires 22, also does not tell the surgeon when sufficient tension has been achieved in the wire 26. This specific method of securing bone halves is plagued with uncertainty in the tension left in its fastener. Tensions left in the wire can vary by 100% even after the surgeon has practiced these specific tensioning techniques.

Thus, it is clear that a fastener that has a tension indicator is of value and is a preferred element of this invention. A buckle modified to reduce friction within the buckle that then abruptly changes to a higher friction state can indicate to the user that an acceptable tension has been achieved within the fastener. In FIG. 6, this would be achieved when the ribbon 58 breaks at the notch 64 to let the pin 56 fall to activate the fastener.

Having a ribbon break to serve as a tension indicator is of value, but only if it can be determined that the ribbon did indeed break. The buckle invented for the specific purpose of securing sternal halves 16 and 18 is quite small. The ribbon designed for this buckle can hardly be seen. Determining whether or not such a small element in a small buckle has broken is an issue. The state of the ribbon needs to be clearly communicated to one of our senses. Communicating this state is an important aspect of the invention.

The ribbon makes a sound when it breaks. It is a weak sound, but can make a clear indication if amplified. A voltage may be passed through the ribbon and monitored by a circuit. Such a circuit can, in turn, make a sound or turn on a light or change the color of a diode if the electrical characteristics change due to the ribbon breaking, or otherwise generate an alarm of some type. A mechanical means might also be connected to the ribbon that serves to indicate that the notch is broken. Such a mechanical means could, as an example, make use of a flag. Such a flag might also be constructed around the ribbon so that it indicates the status of the notch 64. The flag is of a sufficient size so that it may be clearly identified by a surgeon.

The flag operates so that it is secure to the buckle when the notch 64 is intact, and is easily detached from the buckle should the notch 64 break. When designed as such, the surgeon monitors the flag to see if it jumps from the breaking of the notch 64. The surgeon confirms that it is not a false “jump” by testing whether or not the flag is securely attached to the buckle.

The design of the flag is integral to the design of the buckle. The flag has a base, a post, and a large tab. The base facilitates the attachment of the flag to the buckle, and the post raises the tab from the base to be clearly visible by the surgeon. The post and the tab constitute what might be a typical flag. The base is more complex.

In one embodiment, the base has fingers that hold onto the buckle in a very specific manner. The fingers interface with the buckle so that it secures the base to the buckle in every degree of freedom except one. This one degree of freedom that the fingers do not hold must enable the base to easily detach itself freely from the buckle. The one degree of freedom that the fingers do not address (for secure fixation) is addressed by the ribbon/notch construct. The ribbon/notch construct works together with the base fingers to supply secure fixation of the base to the buckle. When the notch breaks due to the appropriate tension in the band, this critical degree of freedom is released, the fingers cannot securely hold the base onto the buckle, and the base's attachment to the buckle becomes unstable as does the flag's attachment to the buckle. This instability serves as an indicator to the surgeon that the desired tension has been met in the band. Designed as such, the flag can communicate to the surgeon the status of the notch 64.

There are a number of related mechanisms that can make use of managing one critical degree of freedom to indicate whether or not the notch 64 has broken. Such a related mechanism appropriates its indicative action to the status of one degree of freedom that is managed by the status of the notch 64.

While the inventive concept is disclosed as being particularly adapted for use in repairing the sternum after a thoracic cavity procedure, it is, of course, applicable to a great many other procedures requiring repair of bodily tissue, particularly bone.

Accordingly, although exemplary embodiments of the invention have been shown and described, it is to be understood that all the terms used herein are descriptive rather than limiting, and that many changes, modifications, and substitutions may be made by one having ordinary skill in the art without departing from the spirit and scope of the invention, which is to be limited only in accordance with the following claims.

What is claimed is:

1. A dynamic tissue holding device for dynamically holding two tissue portions in contact with one another, the device comprising:
a band adapted for extending about the tissue portions to be held together, the band having a first end for attachment to a first end of a frame and a second end for attachment to a second end of a frame;
the band establishing a path of tension along its length and extending linearly between the two ends of the band;
a locking member;
a mating surface disposed on said frame first end;
a restraining member situated to keep the locking member in a spaced relation to the mating surface, the member being constructed to move to a non-restraining orientation when a predetermined level of tension has been achieved in the band; and
a tension indicator which is triggered when the restraining member moves to its non-restraining orientation.

2. The device as recited in claim 1, wherein the restraining member comprises a ribbon having a point of weakness designed therein which is adapted to break at a predetermined tension level.

3. The device as recited in claim 2, wherein the point of weakness comprises a notch in said ribbon.

4. The device as recited in claim 2, wherein said ribbon is attached between said frame and said locking member, said ribbon acting to prevent movement of the locking member toward the mating surface when intact, and further wherein its fracture at the point of weakness when applied tension thereto reaches or exceeds said predetermined tension level permits the locking member to move from its initial position, spaced from said mating surface to a second position engaging said mating surface.

5. The device as recited in claim 2, wherein said ribbon supports said locking member in its initial position, spaced from said mating surface, when intact, and further wherein its fracture at the point of weakness when applied tension thereto reaches or exceeds said predetermined tension level permits the locking member to move along a cam surface to a second engaging position.

6. The device as recited in claim 1, wherein the restraining member comprises a removable pin.

7. The device as recited in claim 1, wherein the frame further comprises a detent and the locking member further comprises a ball engageable with said detent, wherein when the ball and the detent are engaged, they comprise said restraining member.

8. The device as recited in claim 7, wherein said frame is adapted to distort sufficiently, upon application of tension thereto at said predetermined level, to dislodge the ball from the detent, so that the locking member is unrestrained and free to move from its initial position to a second engaging position.

9. The device as recited in claim 1, wherein the tension indicator comprises an electrical circuit which passes a voltage through the restraining member and a sensor for detecting when the voltage changes as a result of the restraining member moving to its non-restraining orientation.

10. The device as recited in claim 9, wherein the sensor activates a visual or audible alarm.

11. The device as recited in claim 1, wherein the tension indicator comprises a mechanical member which is connected to the restraining member.

12. The device as recited in claim 11, wherein the mechanical member comprises a flag which undergoes a visible change when the restraining member moves to its non-restraining orientation.

13. The device as recited in claim 12, wherein the flag detaches from the frame when the restraining member moves to its non-restraining orientation, resulting in a movement thereof which can be visually detected.

14. The device as recited in claim 13, wherein the flag comprises a base, a post, and a tab, the base functioning to connect the flag to the frame and the post raising the tab from the base for ready visibility to a practitioner.

15. The device as recited in claim 14, wherein the base comprises fingers for attaching the base to the frame in every degree of freedom except for one.

16. The device as recited in claim 15, wherein the one degree of freedom not addressed by the attachment of the base to the frame is addressed by the restraining member, such that when the restraining member moves to its non-restraining orientation, the base becomes unstable and detaches from the frame.

17. A method of repairing separated tissues using a tissue tensioning device comprising a locking member, a band having first and second ends, a frame having opposed first and second ends, and a restraining member, wherein the first end of the band is attached to the first end of the frame, the method comprising:
applying tension to the second end of the band so that the band moves freely between spaced engaging surfaces on the locking bar and the second end of the frame, respectively, while the locking member is restrained to a first non-engaging position by a restraining member;
moving the locking member to a second position wherein the engaging surfaces on the locking member and the second side of the frame are sufficiently engaged to clamp the locking member in place, the moving step being performed by applying a predetermined level of tension on the band, so that the restraining member moves to a non-restraining orientation; and
triggering an alarm to notify a practitioner that the predetermined level of tension has been achieved and that the restraining member has thus moved to the non-restraining orientation.

18. The method as recited in claim 17, wherein the triggering step comprises changing an electrical condition in a circuit and thus activating a visual or audible alarm.

19. The method as recited in claim 17, wherein the triggering step comprises changing a state of a mechanical member relative to the device, so that the practitioner can see the changed state of the mechanical member and know that the predetermined tension level has been achieved.
